(19) United States
(12) Patent Application Publication
(10) Pub. No.: US 2025/0291068 A1
(43) Pub. Date: Sep. 18, 2025

(54) GENERATING AN X-RAY IMAGE DATASET BY MEANS OF A PHOTON-COUNTING X-RAY DETECTOR

(71) Applicant: Siemens Healthineers AG, Forchheim (DE)

(72) Inventors: Martin HUPFER, Erlangen (DE); Bjoern KREISLER, Hausen (DE); Bodo REITZ, Forchheim (DE); Edgar GOEDERER, Forchheim (DE); Michael HOSEMANN, Erlangen (DE)

(73) Assignee: Siemens Healthineers AG, Forchheim (DE)

(21) Appl. No.: 19/077,300

(22) Filed: Mar. 12, 2025

(30) Foreign Application Priority Data
Mar. 14, 2024 (DE) 10 2024 202 412.0

(51) Int. Cl. G01T 1/161; G06T 11/00

(52) U.S. Cl. CPC G01T 1/161; G06T 11/003

(57) ABSTRACT

Each pixel element of a plurality of pixel elements has a number of comparators including a first subset of comparators and a second subset of comparators. Each of the second subset of comparators has a threshold value that differs from threshold values of the first subset of comparators. Each pixel element is configured to form at least one count signal based upon the output signal from at least one of the comparators of the first subset. At least a subset of the plurality of pixel elements is configured to form one or more coincidence count signals, wherein at least one coincidence count signal is formed based upon the output signal from at least one of the second subset of comparators of the one pixel element and/or of the at least one further pixel element.

### CROSS-REFERENCE TO RELATED APPLICATION(S)

The present application claims priority under 35 U.S.C. § 119 to German Patent Application No. 10 2024 202 412.0, filed Mar. 14, 2024, the entire contents of which is incorporated herein by reference.

### FIELD

One or more embodiments of the present invention relate to a method for generating an X-ray image dataset via a photon-counting X-ray detector, a photon-counting X-ray detector, an X-ray detector system and a medical imaging device.

### BACKGROUND

In a photon-counting X-ray detector of an X-ray imaging device, X-rays are converted into electrical signal pulses with the aid of a converter material. A photon-counting X-ray detector typically has a plurality of detector elements which are referred to as pixels and which absorb electrical charge carriers generated in the converter material and process them further as electrical signal pulses, using circuitry. A spatial resolution and an image size of the X-ray detector are defined by the size and the distribution of the pixels. The size or the length of an electrical signal pulse is typically proportional to the energy of the absorbed X-ray photon. In this way, an item of spectral information can be extracted through a comparison of the height or length of the electrical signal pulse with an energy threshold. Often, photon-counting X-ray detectors have a plurality of settable energy thresholds for a comparison of the electrical signal pulses generated so that energy-resolved measurements become possible dependent upon a plurality of energy ranges defined by the energy thresholds.

The use of photon-counting X-ray detectors in X-ray imaging offers a series of advantages over energy-integrating X-ray detectors. For example, photon-counting X-ray detectors enable a high spatial resolution and an intrinsically energy-resolved measurement. The image quality of photon-counting X-ray detectors is, however, often limited by the ultimate extent of the charge clouds generated and by the generation of characteristic X-ray radiation in the detector material. This has the result that the entire energy of an X-ray photon is not always deposited in the pixel concerned, but rather that a part of the energy is registered in adjacent pixels. As a result, firstly photons are registered under the wrong energy and, secondly photons can also be counted multiple times in adjacent pixels. This phenomenon is also known as charge sharing. Not only does charge sharing impair the spectral properties of the X-ray detector, but also leads quite generally to a deterioration of the efficiency (also called detective quantum efficiency—DQE) of the X-ray detector, due to an increase in the noise and a decrease in the spatial resolution. This is therefore an effect that can degrade the image quality for all applications.

An approach to solving this problem lies in implementing so-called charge summing circuits on the ASIC (application specific integrated circuit) of the X-ray detector. Herein, during the detection process, it is recognized in the analogue part of the ASIC pixels that charge has been deposited in a plurality of adjacent pixels and the whole charge of all the pixels is assigned to one pixel (typically the pixel with the most charge or the fastest current rise). Thereby, double counting is prevented and the original charge is also almost restored. A disadvantage of such circuits is that the dead time of the pixels is hugely increased by them. Thereby, the problem of the so-called pulse pile-up, whereby the signals of a plurality of photons become overlaid and also lead to falsified measurements, is intensified. A good high flux capability, as required, for example, in computed tomography, is thus in general no longer available.

Alternatively, by way of an increase in the pixel size (e.g. to >0.3 mm edge length), the worsening of the energy resolution and the efficiency level can also be counteracted, although at the cost of the high flux capability (due to the overlaying of individual photon signals as pulse pile-up) and additionally at the cost of the spatial resolving power.

Further approaches are aimed at solving this problem with digital circuitry approaches, in particular, digital summing. Therein, it is not the analogue-measured charges in adjacent pixels that are added, but rather just the digital signals. Thus if a lower energy threshold is exceeded in two adjacent pixels, these two counter events are accumulated to one counter event in a higher threshold, wherein one of the two pixels arbitrarily receives this counter event. This approach has been described, for example, in “Digital count summing vs analogue charge summing for photon counting detectors: A performance simulation study”, Scott Hsieh et al., Med. Phys. 45(9), September 2018. In this approach also, the logic must correct the coincidences in real time, which itself favors an occurrence of negative pile-up effects.

A further approach has been proposed in EP 3 839 577 A1. Therein, apart from the counting of count signals dependent upon the incident X-rays, in each pixel element, coincidence count signals of pixel elements are additionally counted with at least one further pixel element in each case. By this means, it can be made possible to take account of coincidence information thereby obtained in order to lessen a reduction in the image quality due to coincidences arising in the pixel elements of the X-ray detector. The quality of generated X-ray and CT images can thereby be improved. A negative effect on a high flux capacity can also be prevented or reduced. Specific technical implementations for counting coincidence count signals are disclosed, for example, in EP 3 839 578 A1 and EP 3 839 576 A1.

### SUMMARY

Independent of the grammatical term usage, individuals with male, female or other gender identities are included within the term.

It is an object of one or more embodiments of the present invention to find a possibility for further improving the generation of X-ray image datasets, in particular, taking account of coincidences that arise.

At least this object is achieved by a method, a photon-counting X-ray detector, an X-ray detector system and a medical imaging device as claimed. Further features and advantages are disclosed in the dependent claims, the description and the attached drawings.

According to a first aspect of embodiments of the present invention, a method is provided for generating an X-ray image dataset via a photon-counting X-ray detector. The X-ray detector comprises at least one converter element and, connected thereto, a large number of pixel elements, wherein the converter element is configured to convert incident X-ray radiation into an electrical signal, and each of the pixel elements of the large number of pixel elements is configured to record an electrical signal at its respective position, wherein each pixel element of the large number of pixel elements has a number of comparators, each having at least one threshold value, wherein the comparators are each configured to output an output signal if the threshold value is exceeded by the electrical signal, wherein the number of comparators comprises a first subset of comparators and, in the case of at least one subset of the large number of pixel elements, a second subset of comparators, wherein each of the second subset of comparators is set to a threshold value which differs from the threshold values of the first subset of comparators, wherein the pixel elements of the large number of pixel elements are each configured to form at least one count signal based upon the output signal from at least one of the comparators of the first subset, wherein the second subset of comparators is not used for the formation of a count signal, wherein at least the subset of the large number of pixel elements is configured to form one or more coincidence count signals, each of which is based upon the output signal generated in a pixel element of the subset of pixel elements by one of the number of comparators and upon a coinciding output signal generated by one of the number of comparators of at least one further pixel element of the large number of pixel elements, wherein at least one coincidence count signal is formed based upon the output signal from at least one of the second subset of comparators of the one pixel element and/or of the at least one further pixel element, wherein the method comprises the following steps:
first counting of at least one number of count signals dependent upon the incident X-ray radiation in each pixel element of the large number of pixel elements;
second counting of at least one number of coincidence count signals in each pixel element of the subset of the large number of pixel elements with at least one further pixel element of the large number of pixel elements; and
generating an X-ray image dataset based upon the at least one number of count signals counted in each pixel element of the large number of pixel elements and upon the at least one number of coincidence count signals counted in each pixel element of the subset of the large number of pixel elements.

Embodiments of the present invention build upon the knowledge contained in the prior art. The counting of coincidence count signals (second counting) offers the advantage that additional information can be obtained without negatively affecting the actual counting (first counting), in particular without negatively affecting a high flux event. Extended dead times of the pixel elements can be prevented in that a time-consuming real time correction during the measurement itself can be avoided. According to an embodiment of the present invention, by way of the use of the second subset of comparators, further coincidence measurements can be enabled which can be measured, in particular, between or alongside energy levels that are not used simultaneously as threshold values for the first counting. In this regard, the comparators of the second subset of comparators can be regarded as dedicated comparators that are provided only for the second counting of the coincidence count signals. A new idea in embodiments of the present invention, that a suitable or even an optimum energy threshold for the second counting of the coincidence count signals can be different from the suitable or optimum energy threshold(s) for the first counting, extends the possibilities for acquiring coincidence count signals in an advantageous manner. For example, an energy threshold of the coincidence count signal can be optimized separately and without taking account of the energy thresholds or threshold values used for the first counting.

The X-ray image dataset can be in particular an X-ray image dataset of an object and/or subject. An object can be, for example, an anatomical region, for example, comprising an organ or a part of an organ or an object. A subject can be, for example, a human, in particular a patient, or an animal. The photon-counting X-ray detector can be part of an imaging device, in particular, a medical imaging device. The imaging device can be, for example, a computed tomography device.

The converter element can comprise, in particular, a converter material for transforming incident X-ray radiation into the electrical signal. The converter material can be, for example, CdTe, CZT, HgI2, GaAs or another suitable material. The use of CdTe can be particularly advantageous. The pixel elements are configured, in particular, to record an electrical signal generated by the converter element. The pixel elements can also be designated detector elements, image elements, pixels or image cells. The large number of pixel elements can be arranged in a matrix-like pixel grid. The pixel elements can each comprise an electrical circuit, comprising, in particular, the number of comparators. In particular, the electrical circuit can comprise counters for first counting of the number of count signals and, in the case of the subset of the pixel elements, coincidence counters for second counting of the number of coincidence count signals.

Each pixel element of the large number of pixel elements has a number of comparators each having at least one threshold value. The threshold value can be configured to acquire a size, for example, an amplitude of an electrical signal. The threshold value can correspond to an energy threshold. The expression “comparator” is to be understood broadly in the context of this invention. In particular, a comparison member is in general meant, which triggers an output signal if the threshold value as set is exceeded by an incident electrical signal. The threshold value of a comparator can be permanently set. For example, the threshold value can be permanently defined during the manufacturing of the comparator. Alternatively, the threshold value of a comparator can be changeable or settable. For example, the threshold value can be capable of being set to a plurality of permanent threshold values. Alternatively or additionally, the threshold can be settable within a threshold value range.

For the formation of the coincidence count signals, a coincidence logic unit can be provided. The coincidence logic unit can be configured to provide, on occurrence of at least two coincidentally occurring signals, an output signal which can be counted by a counter coupled in a signal carrying manner to the coincidence logic unit as a coincidence count signal.

Optionally, a plurality of count signals can be counted in a respective pixel element of the large number of pixel elements. Optionally, each pixel element can comprise a plurality of comparators of the first subset of comparators. Accordingly, the first counting can be provided for output signals of the plurality of comparators, wherein for each comparator of the plurality of comparators, a number of count signals is counted. For example, in each pixel element of the large number of pixel elements, a plurality of numbers of count signals can be counted dependent upon a plurality of energy threshold values provided for energy-resolving scans. Advantageously, based upon the plurality of numbers of count signals, an energy-resolved X-ray image dataset can advantageously be generated.

Optionally, the second subset of comparators can comprise exactly one comparator. Alternatively, each pixel element of the subset of the large number of pixel elements can comprise a plurality of comparators of the second subset of comparators. In this way, a particularly flexible adaptation to the circumstances of a particular scan can be possible.

Optionally, a plurality of counts of coincidence count signals can be counted in a respective pixel element of the subset of the large number of pixel elements. The counting of a plurality of numbers of coincidence count signals can be provided, in particular, based upon the output signals from a plurality of comparators of the number of comparators of the respective pixel element of the subset of the large number of pixel elements, and at least upon the output signal of a comparator of the number of comparators of the at least one further pixel element of the large number of comparators. The counting of a plurality of coincidence count signals can enable more detailed coincidence information, in particular, dependent upon a plurality of energy thresholds. By this mechanism and/or means, the possibilities of a correction of the inaccuracies caused by coincidences can be improved.

Optionally, the subset of the large number of pixel elements can thereby comprise the whole large number of pixel elements. The large number can, however, also comprise, apart from the subset of pixel elements, differently configured pixel elements. These can be configured, for example, merely to form and to count count signals. Since only a part of the pixel elements of the large number of pixel elements is configured to form and count coincidence count signals, for example, a simplified circuit arrangement of the pixel elements can be enabled.

The at least one further pixel element of the large number of pixel elements on which a coincidence count signal to be counted is based can be included by the subset of the large number of pixel elements, i.e. it can itself be part of the subset of the large number of pixel elements. However, embodiments can also exist in which the at least one further pixel element is not part of the subset of the large number of pixel elements.

At least one coincidence count signal is formed based upon an output signal from at least one of the second subset of comparators. Optionally, a plurality of coincidence count signals is formed based upon an output signal from at least one of the second subset of comparators.

For the generation of an X-ray image dataset, the at least one number of coincidence count signals can be incorporated, for example, into the data preprocessing before an image reconstruction, into the image reconstruction and/or into a postprocessing step downstream of the image reconstruction. For example, at least in each pixel element of the subset of the large number of pixel elements, the at least one number of coincidence count signals can be subtracted from the at least one number of count signals or added to the at least one number of coincidence count signals. A subtraction or addition can thereby comprise a weighted subtraction or addition. This means that just a portion or a multiple of the at least one number of coincidence count signals can be subtracted or added. However, other implementations can exist, by which at least one number of count signals can be adapted. If a plurality of numbers of count signals are counted in the pixel elements, for example, dependent upon a plurality of energy thresholds, each or only a part of the numbers can be adapted with the aid of the established at least one number of coincidence count signals. If, similarly, a plurality of numbers of coincidence count signals is established, for example, dependent upon a plurality of energy thresholds, different numbers of coincidence count signals can be applied to different numbers of count signals for an adaptation.

According to one embodiment, the output of the comparators of the first subset of comparators is directly or indirectly connected to at least one counter for the first counting of count signals, wherein the output of the comparators of the second subset of comparators is not connected to a counter for first counting of count signals. Since the second subset of comparators is not used for the formation of a count signal, advantageously, the circuitry components needed therefor can also be dispensed with. In particular, connecting counters for the first counting of the number of count signals to the comparators of the second subset of comparators can also be dispensed with. Since such components, in particular counters, can be relatively demanding of space due to their size, a space saving can thus be achieved. Despite this space saving, by way of the second subset of comparators, threshold values for the second counting can also be used which are not present at all as threshold values for the first counting.

According to one embodiment, the threshold value of the at least one of the second subset of comparators is set so that a special information item that is relevant for a scan and/or examination can be acquired in a targeted manner, in particular such that the information item can be better acquired than would be possible just with the threshold values of the first subset of comparators. For example, a threshold value can be used that is lower than the threshold values of the first subset of comparators. Advantageously thereby, it can also be acquired if portions of an X-ray photon are distributed over a plurality of pixel elements, so that some portions are below the lowest threshold value of the first subset of comparators. This low threshold is possibly not relevant for the actual counting of the events, although it can be relevant for the acquisition of coincidences. Accordingly, other threshold values can be useful specifically for the establishment of coincidences. In the case of a comparator with a settable threshold value, an additional setting step can be provided. It can be provided to set the threshold value of the at least one of the second subset of comparators in a targeted manner so that an information item specific to a scan and/or examination can be acquired in a targeted way. It can be provided that the determination of a plurality of events leading to coincidence is combined, in particular in that a plurality of numbers of coincidence events are counted in the respective pixel element. For example, both a symmetrical and also an asymmetrical coincidence can be acquired and taken into account. Advantageously, therefore, the respective effects of the coincidence can possibly be broken down.

According to one embodiment, the threshold value of the at least one of the second subset of comparators is adapted to a property, in particular a material property, of an object to be examined and/or to a property, in particular a material property, of the converter element. Advantageously, therefore, coincidence counting can be adapted to the respective current scan. A property of the object to be examined can be, for example, an, in particular, energy-dependent absorption coefficient.

According to one embodiment, the threshold value of the at least one of the second subset of comparators is adapted to an X-ray spectrum of an X-ray source used for the recording of the X-ray image dataset.

According to one embodiment, the threshold value of the at least one of the second subset of comparators is set relative to at least one fluorescence energy of a material of the converter element. In particular, the threshold value of the at least one of the second subset of comparators can be set in such a way that the threshold value of the at least one of the second subset of comparators and a lowest threshold value of the first subset of comparators that is provided, in particular, for first counting, are on different sides of the fluorescence energy. In other words, a lowest threshold value of the first subset of comparators can be below the fluorescence energy and the threshold value of the at least one of the second subset of comparators can be above the fluorescence energy or a lowest threshold value of the first subset of comparators can be above the fluorescence energy and the threshold value of the at least one of the second subset of comparators can be below the fluorescence energy. For example, given a fluorescence energy of 23 keV, one of the specified threshold values can be at 20 keV and the other threshold value at 25 keV. It can be provided that the threshold value of the at least one of the second subset of comparators is between the fluorescence energy and the next of the threshold values of the first subset of comparators is on this side of the threshold value and/or that the threshold value is closer to the fluorescence energy than each of the threshold values of the first subset of comparators. Fluorescence is an effect in which X-ray photons initially excite the material of the converter elements and shortly thereafter are spontaneously emitted again. If individual photons have a sufficient energy, for example, above a K-edge of the converter material, then fluorescence can occur. Due to the mean free path length of the re-emitted photons (typically in the order of approximately 100 μm), the photon can often reach an adjacent pixel element, which is adjacent to the pixel element at which the X-ray photon originally arrived. There, the photon is then reabsorbed in the adjacent pixel element and is possibly registered by way of the first counting. The case of reabsorption in an adjacent pixel element can bring with it the consequence that two counter events occur and the energy of the primary X-ray photon is distributed over both pixel elements. Thus both the counting rate and also the respectively detected energies are faulty. The smaller the pixel elements are made, the more often this case applies. Typically, the reabsorbed photon has a lower energy than the originally incident X-ray photon. In the context of this invention, it has been recognized that the threshold values used for the first counting of the at least one number of count signals are not necessarily well suited to determining fluorescence effects optimally. Advantageously, with this embodiment, the effect of fluorescence can be specifically taken into account, for example, with regard to the evaluation of the measurement data. In the case of a comparator with a settable threshold value, an additional setting step can be provided. It can be provided to set the threshold value of the at least one of the second subset of comparators in a targeted manner relative to at least one fluorescence energy of the material of the converter element.

According to one embodiment, the threshold value is above or below all the substantial fluorescence energies of the material of the converter element. It can be provided, for example, that the threshold value of the at least one of the second subset of comparators is below the fluorescence energy. In particular, where relevant, the threshold value is also above the next lower threshold value of the first subset of comparators. Coincidences that are based upon fluorescence photons can thus be specifically acquired. It can be provided that the threshold value of the at least one of the second subset of comparators is above the fluorescence energy. For example, therefore, coincidences that are not based upon the corresponding fluorescence effect can be specifically sought. Coincidences occur, for example, when an ultimate extent of the charge cloud has the result that the energy of an X-ray photon is deposited in a plurality of adjacent pixel elements (also called charge sharing). It is possible, in particular as described herein, by way of asymmetrical acquisition, to establish more precise positional information for an incident X-ray photon. However, in order not to confuse the effects of fluorescence and charge sharing, it can, for example, be useful specifically to avoid the acquisition of fluorescences and, for example, to place a threshold value just above the fluorescence energy.

According to one embodiment, the threshold value is between two fluorescence energies of the material of the converter element. For example, therefore, individual fluorescence thresholds can be specifically looked for.

According to one embodiment, for the second counting of the at least one number of coincidence count signals, the threshold value of the at least one comparator of the number of comparators of the respective pixel element of the subset of the large number of pixel elements and the settable energy threshold of the at least one comparator of the number of comparators of the at least one further pixel element of the large number of pixel elements on which the coincidence count signals are based, has an identical energy threshold value. Advantageously, therefore, coincidence count signals can be counted which are based upon coincidingly occurring signals which, both in the respective pixel element under consideration as well as in the at least one further pixel element, each have exceeded the same energy threshold value. This can also be referred to as a symmetrical acquisition of coincidences.

According to one embodiment, for the second counting of the at least one number of coincidence count signals, the threshold value of the at least one comparator of the number of comparators of the respective pixel element of the subset of the large number of pixel elements and the threshold value of the at least one comparator of the number of comparators of the at least one further pixel element, on which the coincidence count signals are based, different threshold values are set. This can also be referred to as asymmetrical acquisition of coincidences. Optionally, both an asymmetrical acquisition and also a symmetrical acquisition can be provided in that accordingly, at least two numbers of coincidence count signals are counted. The asymmetrical acquisition can be advantageous to assign an X-ray photon to one of two pixel elements and/or in order to enable a proportional counting with a mixing factor. The use of different energy thresholds for the threshold values permits, for example, the targeted measurement of coincidence events wherein the minimum value of the deposited energy in the pixel under consideration is higher than the minimum value of the neighbors. In this way, coincidence events are counted in a statistically preferred manner in the pixel in which the highest energy deposition occurs. This improves the assignment of the hits to the pixels and thus the spatial resolution. By way of the use of the second subset of comparators, the threshold values for the asymmetrical acquisition can be selected particularly flexibly.

According to one embodiment, a coincidence count signal is counted on a pixel element if it is detected that in this pixel element, coincidentally a higher threshold value of a comparator is exceeded than in the at least one further pixel element and/or wherein, dependent upon a highest exceeded threshold value on the pixel element and an acquired highest exceeded threshold value on the at least one further pixel element, a coincidence count signal is processed with a mixing factor. A plurality of similar asymmetrical events can be acquired, in particular, to establish in which pixel element a largest portion of an X-ray photon has been deposited. The mixing factor can be provided to adapt the counted value according to the portion of the X-ray photon that has been deposited in the respective pixel element. If, for example, a third of the energy of the X-ray photon has been deposited in the detector element, the mixing factor could be, for example, one third. Gradations of the mixing factor can be provided according to the threshold values used and the accuracy possible therewith. It can be provided that the mixing factor has a value of less than 1. It can be provided that the mixing factor is adapted to a relative height of the threshold values of the asymmetrical acquisition and/or to a relative energy content of adjacent coincident events. It can be provided, for example, that mixing factors are provided for all the pixel elements that have a common coincidence event. The sum of the mixing factors can be set, for example, to 1. For example, the mixing factor can be 0.4 for a first pixel element and 0.6 for a second pixel element. For example, the threshold value of the respective pixel element of the subset of the large number of pixel elements can be higher than the threshold value of the at least one further pixel element of the large number of pixel elements. It can thus be provided that coincidence events are counted in a statistically preferred manner in the pixel in which the highest energy deposition occurs. This can improve the assignment of the hits to the pixel elements and thus the spatial resolution. For example, the coincidence signal of a 30 keV threshold can be measured with coincidence signals of the 20 keV threshold of the adjacent pixel. It can be provided, in addition to an asymmetrical acquisition for specifying a location, to provide an acquisition of fluorescences and/or further coincidence-induced events. Advantageously, therefore, the respective effects of the coincidence can possibly be broken down.

According to one embodiment, it is provided that in each pixel element of the subset of pixel elements, both at least one quantity of coincidence count signals which is based upon the same energy threshold value is counted, and at least one number of coincidence count signals is counted which is based upon different energy threshold values. This embodiment can enable a particularly detailed coincidence information.

According to one embodiment, at least one of the number of comparators is a switchable comparator, the threshold value of which is settable to different threshold values. The at least one switchable comparator can be a comparator of the first subset of comparators. In addition or alternatively, the at least one switchable comparator can be a comparator of the second subset of comparators. A plurality of switchable comparators can be provided. Optionally, all of the number of comparators can be provided to be switchable. By way of a switchable comparator, the counting of coincidence count signals can be adapted particularly flexibly to the respective circumstances of a scan. A further method step can be provided: Setting the at least one of the number of comparators, in particular, at least one comparator of the second number of comparators, to a threshold value. The setting can take place, for example, automatically based upon a scan protocol. Alternatively, the acquisition of a user input can be provided with which the threshold value to be set is specified. By way of example, an asymmetry of two comparators that are used for second counting of a number of coincidence count signals can be adapted by a setting of the at least one switchable comparator. By way of example, in some cases it can be advantageous to use a threshold value for the second counting of coincidence count signals that is higher than the lowest threshold value for the first counting of count signals. By way of example, a threshold value can be adapted with regard to a focus on a higher spectral resolution and/or with a focus on a higher spatial resolution. In this event, for example, a high threshold value and/or a high elimination of count signals can be advantageous. If it is an aim to have the highest possible signal intensity, the use of lower threshold values for counting count signals can be advantageous. Optionally, the threshold value of the at least one of the second subset of comparators can be set so that a specific information item relevant to a scan and/or examination can be acquired in a targeted way. Optionally, a switchable comparator of the respective pixel element of the subset of the large number of pixel elements can be provided. Optionally, a comparator of the at least one further pixel element of the large number of pixel elements can additionally have a fixed threshold value, in particular a lowest threshold value. For example, with a circuit of this type, the measurement of coincidences of the type a/a and b/a (e.g. 20/20 keV and 30/20 keV) can be enabled.

According to one embodiment, the at least one further pixel element comprises an adjacent pixel element. In particular, for the second counting, the at least one further pixel element of the large number of pixel elements can be adjacent to the respective pixel element of the subset of the large number of pixel elements. Adjacent should be understood to mean that no further pixel element is arranged between two pixel elements. By way of example, in a rectangular matrix of pixel elements, pixel elements can be immediately or diagonally adjacent to one another.

According to one embodiment, for each pixel element of the large number of pixel elements, in particular either on the pixel element itself or on another pixel element, at least a number of coincidence count signals can be established. By way of example, a number of coincidence count signals can be established on a pixel element and then also used for at least one further pixel element in the vicinity. “In the vicinity” can mean, for example, adjacent. “In the vicinity” can mean, for example, within a similar subgroup of pixel elements. A subgroup can preferably consist of a coherent matrix of not more than 40, preferably not more than 30 pixel elements.

A further aspect of embodiments of the present invention is a photon-counting X-ray detector, which comprises at least one converter element and connected thereto a large number of pixel elements and counters for counting count signals and coincidence counters for counting coincidence count signals, wherein the converter element is configured to convert incident X-ray radiation into an electrical signal, and each pixel element of the large number of pixel elements is configured to record an electrical signal at its respective position, wherein each pixel element of the large number of pixel elements has a number of comparators, each having at least one threshold value, wherein the comparators are each configured to output an output signal if the threshold value is exceeded by the electrical signal, wherein the number of comparators comprises a first subset of comparators and, in the case of at least one subset of the large number of pixel elements, a second subset of comparators, wherein the pixel elements of the large number of pixel elements are each configured to form at least one count signal based upon the output signal from at least one of the comparators of the first subset, wherein at least the subset of the large number of pixel elements is configured to form one or more coincidence count signals, each of which is based upon the output signal generated in a pixel element of the subset of pixel elements by one of the number of comparators and upon a coinciding output signal generated by one of the number of comparators of at least one further pixel element of the large number of pixel elements, wherein the output of each of the comparators of the first subset of comparators is connected directly or indirectly to at least one counter for counting count signals of individual electrical signals and optionally to at least one coincidence counter for counting coincidence signals of coincident electrical signals, wherein the output of each of the comparators of the second subset of comparators is connected to at least one coincidence counter for counting coincidence signals of coincident electrical signals and not to a counter for counting count signals of individual electrical signals. All the advantages and features of the method can be transferred likewise to the X-ray detector and vice versa. Optionally, the first subset of comparators can comprise a plurality of comparators. For example, the first subset of comparators can comprise 1 to 10 comparators, preferably 2 to 8 comparators. For example, the second subset of comparators can comprise 1 to 5 comparators, preferably 1 to 3 comparators. Optionally, a subset of pixel elements can comprise the whole large number of pixel elements. The X-ray detector can be configured, in particular, to carry out the method steps of the first counting and of the second counting according to the method as described herein.

According to one embodiment, at least one of the number of comparators of the X-ray detector is a switchable comparator, the threshold value of which is settable to different threshold values. In particular, at least one of the second subset of comparators can be a switchable comparator. Optionally, a plurality, or all, of the number of comparators can be a switchable comparator. In particular, the features and advantages of the settable comparators as herein described elsewhere can be applied to this embodiment.

For example, it can be advantageous to adapt the at least one of the threshold values of the comparators to a respectively used operating mode or dependent upon the scan protocol used for the X-ray detector, or an imaging device in which the X-ray detector is used. For example, dependent upon the operating mode or scan protocol, a pile-up effect can occur sooner or more probably. Accordingly, a minimum threshold for the second counting of the coincidence count signals can be adapted.

A further aspect of embodiments of the present invention is an X-ray detector system which is configured to carry out a method as described herein. The X-ray detector system comprises a photon-counting X-ray detector, in particular as described herein, comprising at least one converter element and connected thereto a large number of pixel elements, wherein the converter element is configured to convert incident X-ray radiation into an electrical signal, and each pixel element of the large number of pixel elements is configured to record an electrical signal at its respective position, wherein each pixel element of the large number of pixel elements has a number of comparators, each having at least one threshold value, wherein the comparators are each configured to output an output signal if the threshold value is exceeded by the electrical signal, wherein the number of comparators comprises a first subset of comparators and, in the case of at least one subset of the large number of pixel elements, a second subset of comparators, wherein the pixel elements of the large number of pixel elements are each configured to form at least one count signal based upon the output signal from at least one of the comparators of the first subset, wherein at least the subset of the large number of pixel elements is configured to form one or more coincidence count signals, each of which is based upon the output signal generated in a pixel element of the subset of pixel elements by one of the number of comparators and upon a coinciding output signal generated by one of the number of comparators of at least one further pixel element of the large number of pixel elements. The X-ray detector system further comprises a generating unit, configured to generate an X-ray image dataset based upon at least one number of count signals counted in each pixel element of the large number of pixel elements and upon at least one number of coincidence count signals counted in each pixel element of the subset of the large number of pixel elements. All the advantages and features of the method and of the X-ray detector can be transferred likewise to the X-ray detector system and vice versa. Optionally, the subset of pixel elements can comprise the whole large number of pixel elements.

A further aspect of embodiments of the present invention is a medical imaging device comprising an X-ray detector system as described herein. The medical imaging device can comprise, for example, a computed tomography device, a C-arm X-ray device or an angiography X-ray device. However, other medical imaging devices which are configured to generate a two-dimensional or a three-dimensional image dataset of an object or subject, in particular a patient, based upon X-ray radiation are also possible. All the advantages and features of the method, of the X-ray detector and of the X-ray detector system can be transferred likewise to the imaging device and vice versa.

All the embodiments described herein can be combined with one another if not explicitly stated otherwise.

### BRIEF DESCRIPTION OF THE DRAWINGS

Embodiments will now be described making reference to the accompanying drawings.

FIG. 1 shows a circuit diagram of a pixel element 1 according to one embodiment of the present invention,

FIG. 2 shows a flow diagram of a method for generating an X-ray image dataset via a photon-counting X-ray detector according to one embodiment of the present invention, and

FIG. 3 shows a medical imaging device having an X-ray detector system according to one embodiment of the present invention.

### DETAILED DESCRIPTION

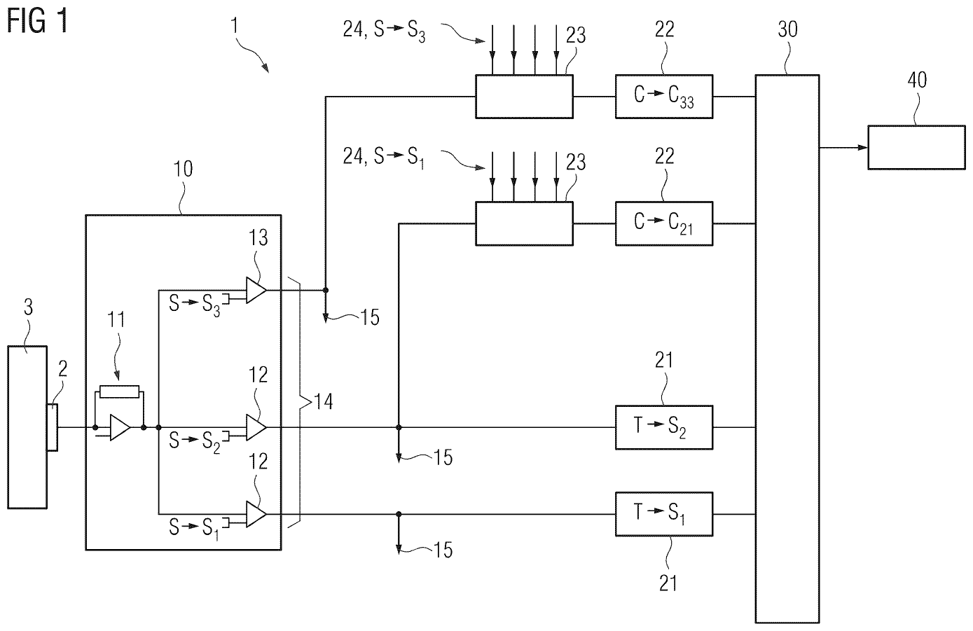

FIG. 1 shows a circuit diagram of a pixel element 1 according to one embodiment of the present invention. The pixel element 1 is connected via an electrode 2 to a converter element 3. On an opposite side of the converter element 3, a corresponding counterelectrode (not shown here) is typically provided. Incident X-ray radiation is converted in the converter material of the converter element 3, dependent upon the energy locally deposited by the incident X-ray radiation, into charge carriers, so that an electrical signal can be tapped off with the aid of the electrodes 2. The pixel element 1 connected at the relevant site can thus record and further process an electrical signal, typically in the form of an electrical pulse. For the further processing of the electrical pulse, the pixel element 1 has a conversion apparatus 10. The conversion apparatus can optionally comprise a signal amplifier 11. Furthermore, the pixel element 1 has a number of comparators 12, 13. In this example, the number of comparators 12, 13 includes three comparators 12, 13. However, a different number can also be provided. Also, another number of count signals T and/or coincidence count signals C can be counted than is mentioned here by way of example.

The conversion apparatus 10 is coupled in a signal carrying manner, optionally via the signal amplifier 11, to the part of the converter element 3 assigned to the pixel element 1, via the electrode 2. The signal amplifier 11 can optionally amplify the electrical signal directly entering the pixel element 1 via the electrode 2 and generated by way of incident X-ray radiation via the converter element 3 for the subsequent further processing. The comparators 12, 13 of the pixel element 1 shown each have a settable threshold value S for setting an energy threshold. The settable threshold values S of different comparators 12, 13 can be set to different energy threshold values. The different threshold values S are identified by the designations S1, S2 and S3. Two of the comparators 12 belong to a first subset of comparators 12 and a third of the comparators 13 belongs to a second subset of comparators 13. The set threshold value S3 of the comparator 13 of the second subset of comparators 13 differs from the set threshold values S1, S2 of the first subset of comparators 12.

Typically, corresponding threshold values S of different pixel elements 1 of the large number of pixel elements 1 can each be set to the same respective threshold values S. However, other embodiments of the threshold values S can also exist. Via the comparators 12, 13, the electrical signal directly entering the pixel element 1 under consideration via the electrode 2 is compared with the respective energy threshold values S→S1, S→S2 and S→S3 of the comparators 12, 13. If a corresponding energy threshold S is exceeded, an output signal which serves as a count signal or coincidence count signal is formed at the signal output 14 of that comparator 12, 13 the energy threshold value S of which has been exceeded. If a plurality of threshold values S are exceeded, a respective count signal is formed at all the signal outputs of the comparators 12, 13 at which a threshold value S has been exceeded.

Based upon the output signal at the signal output 14 of the comparators 12 of the first subset of comparators 12, two numbers T of count signals are counted. The output signal from one of the comparators 12 of the first subset of comparators 12 and of the comparator 13 of the second subset of comparators 13 are also used for counting two numbers of coincidence count signals C. For this purpose, in each case, at least one output signal of a comparator 12, 13 of the number of comparators 12, 13 of at least one further pixel element 1 is still called upon.

In the exemplary embodiment shown, the signal outputs 14 of the comparators 12 of the first subset of comparators 12 are each linked to a counter 21 in a signal carrying manner. The signal outputs 14 of one of the comparators 12 of the first subset of comparators 12 and of the comparators 13 of the second subset of comparators 13 are each linked in a signal carrying manner to a coincidence counter 22. The counters 21 are each configured to count a number T of count signals based upon the output signal of a respectively linked comparator 12. In this exemplary embodiment, one of the counters 21 counts a number T→T1 of count signals dependent upon the threshold value S→S1 and a further counter 21 counts a number T→T2 of count signals dependent upon the threshold value S→S2. Thus, electrical pulses which enter into the converter element 3 are registered as a counting event in the pixel element 1 and counted as a count signal if the electric pulse generated in the pixel element 1 is above the set threshold value S, i.e. above an energy threshold. In this case, the counter level of the counter 21 linked thereto is incremented by one count unit.

Furthermore, the comparators 12, 13 are each linked with the threshold values S→S2 and S→S3, to a coincidence logic unit 23, in a signal carrying manner. In other embodiments, only one or further coincidence logic units 23 can be provided. In each case, however, it is provided that at least one of the comparators 13 of the second subset of comparators 13 (in this example the only comparator of the subset of comparators 13) is linked to at least one of the coincidence logic units 23.

The coincidence logic units 23 are each configured to form a coincidence count signal which is based upon the signal directly entering the pixel element 1 of the subset of pixel elements 1, and upon a coincidentally occurring signal of at least one further pixel element 1 of the large number of pixel elements 1. The respective coincidence logic unit 23 is also linked for this purpose to at least one further pixel element 1 of the large number of pixel elements 1 via at least one further signal input 24 of the respective coincidence logic unit 23. In particular, a respective coincidence logic unit 23 is configured according to a preferred embodiment, based upon the output signal of the respective comparator 12, 13 of the pixel element 1 under consideration, said comparator being coupled to the coincidence logic unit 23, and based at least upon the output signal of a comparator 12, 13 of at least one further pixel element 1 (not shown here), to form a coincidence count signal and to output it at a signal output of the coincidence logic unit 23. In the exemplary embodiment described here, a respective coincidence logic unit 23 is linked, by way of example, to four further pixel elements 1 of the large number of pixel elements 1 in a signal carrying manner and, for this purpose, has four signal inputs 24.

For example, the four further pixel elements 1 can comprise the four directly adjacent pixel elements 1 of the pixel element 1 under consideration in a matrix-like arrangement of the large number of pixel elements 1. Based upon the output signal of the respective coincidence logic unit 23, a number C of coincidence count signals can then be counted with each respective coincidence counter 22.

In the exemplary embodiment, the lower of the coincidence logic units 23 is linked to comparators 12 of the first subset of comparators 12 of the respective further pixel elements 1 which have the energy threshold S→S1. Accordingly, the number C of coincidence count signals counted by the coincidence counter 22 linked to this coincidence logic unit 23 corresponds to the number of coincidentally occurring signals which have exceeded the threshold value S→S2 in the pixel element 1 under consideration and, in at least one of the further pixel elements 1 linked to the coincidence logic unit 23, have exceeded the threshold value S→S1. Thus, a number C→C21 of coincidence count signals is counted based upon different threshold values of the respectively involved pixel elements 1. This can also be referred to as asymmetrical coincidence. Asymmetrical coincidence can be used, for example, to improve a spatial resolution. For example, it can be provided to count a coincidence count signal on a pixel element 1 only if it is detected that in this pixel element 1, coincidentally a higher threshold value of a comparator 12, 13 is exceeded than in the at least one further pixel element 1 with which a comparison is made. Alternatively or additionally, the use of a mixing factor which is determined dependent upon at least one asymmetrical coincidence can also be provided.

The upper of the coincidence logic units 23 is linked, in this exemplary embodiment to comparators 13 of the second subset of comparators 13 of the respective further pixel elements 1 which have the energy threshold S→S3. Accordingly, the number C of coincidence count signals counted via the coincidence counter 22 linked to this coincidence logic unit 23 corresponds to the number of coincidentally occurring signals which have each exceeded the threshold value S→S3 in the pixel element 1 under consideration and, in at least one of the further pixel elements 1 linked to the coincidence logic unit 23. Thus, a number C→C33 of coincidence count signals is counted based upon the same threshold values of the respectively involved pixel elements 1. This can also be referred to as symmetrical coincidence.

Furthermore, in the example shown in FIG. 1, provided coupled to each of the comparators 12, 13, is an output 15, by which a corresponding output signal can be output at one or a plurality of further pixel elements 1 of the subset of pixel elements 1 in order to serve equally as an input signal of a coincidence logic unit 23 of one of the further pixel elements 1 (not shown here) of the subset of pixel elements 1.

The numbers T of count signals and the numbers C of coincidence count signals can be read out from the counters 21 and the coincidence counters 22 via a readout element 30 and output to a generating unit 40 for the generating of the X-ray image dataset. For this, the readout element 30 can be connected via connections and/or via a peripheral electronics system to the generating unit 40.

In general, in the context of embodiments of the present invention, dependent upon the circuit arrangement of the pixel element 1 under consideration of the subset of pixel elements 1, at least one quantity C→Cnm of coincidence count signals can be counted in the pixel element 1 under consideration, wherein n∈{1, . . . , N} with the number N of the provided threshold values S→Sn in the pixel element 1 under consideration and wherein m∈(1, . . . , M) with the number M of provided threshold values S→Sm of the at least one further pixel element 1 of the large number of pixel elements 1 upon which the coincidence count signals are based. Therein, at least one threshold value S is used which is not also used for the first counting of count signals. Preferably, a plurality of numbers C→Cnm of coincidence count signals are counted in each pixel element 1 of the subset of pixel elements 1. Advantageously, a plurality of counted numbers of coincidence count signals permits a further-reaching, improved correction of the X-ray image dataset. For the acquisition of the coincidence count signals of all the pixel elements 1 of the subset of pixel elements 1, for example, a tensor Ĉ can be provided which comprises the numbers Cnm for each of the subset of pixel elements 1, for example, in the form Ĉ=Cₙₘ^{p}, with the subset p of the pixel elements 1.

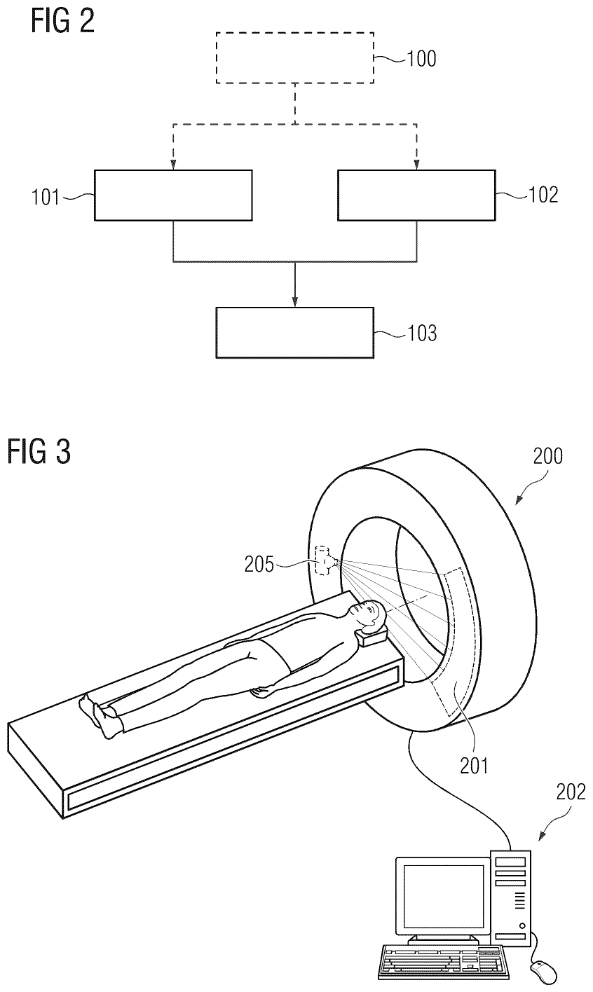

FIG. 2 shows a flow diagram of a method for generating an X-ray image dataset via a photon-counting X-ray detector 201 according to one embodiment of the present invention. The method can be carried out, in particular, with an X-ray detector 201 having pixel elements 1 that are configured according to FIG. 1. The X-ray detector 201 comprises at least one converter element 3 and connected thereto a large number of pixel elements 1. The converter element 3 is configured to convert incident X-ray radiation into an electrical signal and each of the large number of pixel elements 1 is configured to record an electrical signal at its respective position. Each pixel element 1 of the large number of pixel elements 1 has a number of comparators 12, 13, each having at least one threshold value, wherein the comparators 12, 13 are each configured to output an output signal if the threshold value is exceeded by the electrical signal. The number of comparators 12, 13 comprises a first subset of comparators 12 and, in the case of at least one subset of the large number of pixel elements 1, a second subset of comparators 13. The second subset of comparators 13 is each set to a threshold value which differs from the threshold values of the first subset of comparators 12. The large number of pixel elements 1 are each configured to form at least one count signal based upon the output signal from at least one of the comparators 12 of the first subset, wherein the second subset of comparators 13 is not used for the formation of a count signal. At least the subset of the large number of pixel elements 1 is configured to form one or more coincidence count signals, each of which is based upon the output signal generated in a pixel element 1 of the subset of pixel elements 1 by one of the number of comparators 12, 13 and upon a coinciding output signal generated by one of the number of comparators 12, 13 of at least one further pixel element 1 of the large number of pixel elements 1. At least one coincidence count signal is formed based upon the output signal from at least one of the second subset of comparators 13 of the one pixel element 1 and/or of the at least one further pixel element 1. For example, the threshold value of the at least one of the second subset of comparators 13 can be set so that a specific information item relevant for a scan and/or examination can be acquired in a targeted way. In particular, the information can thus be better acquired than would be possible only with the threshold values of the first subset of comparators 12. For example, the threshold value of the at least one of the second subset of comparators 13 can be set in a targeted manner relative to at least one fluorescence energy of the material of the converter element 3, in order thereby also to be able to take account of the influence of fluorescences.

In a first step 101 of the method, a first counting of at least one number of count signals takes place dependent upon the X-ray radiation incident in each pixel element 1 of the large number of pixel elements 1.

In a further step 102, a second counting of at least one number of coincidence count signals takes place in each pixel element 1 of the subset of the large number of pixel elements 1 with at least one further pixel element 1 of the large number of pixel elements 1. These two steps 101, 102 are preferably carried out substantially simultaneously.

In a further step 103, which follows, in particular, upon the two other steps, a generation of an X-ray image dataset is provided based upon the at least one number of count signals counted in each pixel element 1 of the large number of pixel elements 1 and upon the at least one number of coincidence count signals counted in each pixel element 1 of the subset of the large number of pixel elements 1.

Optionally, at the beginning of the method, a step 100 of setting a threshold value S of at least one settable comparator can be provided. This setting can be undertaken, for example, dependent upon a scan protocol that is provided. Optionally, all the threshold values S can be set at the beginning of the method.

FIG. 3 shows a medical imaging device, specifically a computed tomography device having an X-ray detector system according to one embodiment of the present invention. The X-ray detector system comprises a gantry with an X-ray radiation source 205 and an X-ray detector 201 which is part of the X-ray detector system. A generating unit 40 can be arranged, for example, externally to the gantry. It is also conceivable that the generating unit 40 is integrated on the gantry 200 or in the gantry. The generating unit 40 can optionally also be spatially separated from the gantry 200 at another location. For control of the imaging device, a computing unit 202 is herein provided. For example, the generating unit 40 of the X-ray detector system according to embodiments of the present invention can also be included by the computing unit 202.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAN), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A method for generating an X-ray image dataset via a photon-counting X-ray detector, wherein the photon-counting X-ray detector includes at least one converter element connected to a plurality of pixel elements, wherein the at least one converter element is configured to convert incident X-ray radiation into an electrical signal and each pixel element of the plurality of pixel elements is configured to record an electrical signal at a respective position, wherein each pixel element of the plurality of pixel elements has a number of comparators, each comparator having at least one threshold value and each comparator being configured to output an output signal in response to the threshold value being exceeded by the electrical signal, wherein the number of comparators includes a first subset of comparators and, in case of at least one subset of the plurality of pixel elements, a second subset of comparators, wherein each comparator of the second subset of comparators is set to a threshold value that differs from threshold values of comparators of the first subset of comparators, wherein each pixel element of the plurality of pixel elements is configured to form at least one count signal based upon the output signal from at least one comparator of the first subset of comparators, wherein the second subset of comparators is not used for forming a count signal, wherein at least the subset of the plurality of pixel elements is configured to form one or more coincidence count signals, each coincidence count signal based upon the output signal generated in a pixel element of the subset of the plurality of pixel elements by one of the number of comparators, and based upon a coinciding output signal generated by one of the number of comparators of at least one further pixel element of the plurality of pixel elements, wherein at least one coincidence count signal is formed based upon the output signal from at least one comparator of the second subset of comparators of at least one of the pixel element or the at least one further pixel element, wherein the method comprises:
first counting a number of count signals based on the incident X-ray radiation in each pixel element of the plurality of pixel elements;
second counting a number of coincidence count signals in each pixel element of the subset of the plurality of pixel elements with at least one further pixel element of the plurality of pixel elements; and
generating an X-ray image dataset based on the number of count signals counted in each pixel element of the plurality of pixel elements and the number of coincidence count signals counted in each pixel element of the subset of the plurality of pixel elements.

2. The method as claimed in claim 1, wherein
the output of the comparators of the first subset of comparators is directly or indirectly connected to at least one counter for the first counting of the number of count signals, and
the output of the comparators of the second subset of comparators is not connected to a counter for the first counting of the number of count signals.

3. The method as claimed in claim 1, wherein
the threshold value of the at least one comparator of the second subset of comparators is set so that a special information item that is relevant for at least one of a scan or examination is acquired in a targeted manner.

4. The method as claimed in claim 1, wherein the threshold value of the at least one comparator of the second subset of comparators is set relative to at least one fluorescence energy of a material of the converter element.

5. The method as claimed in claim 1, wherein for the second counting of the number of coincidence count signals, the threshold value of at least one comparator of a number of comparators of a respective pixel element of the subset of the plurality of pixel elements and the threshold value of at least one comparator of a number of comparators of the at least one further pixel element, upon which the coincidence count signals are based, are set to different threshold values.

6. The method as claimed in claim 5, wherein at least one of
a coincidence count signal on the respective pixel element is counted in response to detecting, at the respective pixel element, that a higher threshold value of a comparator is exceeded than in the at least one further pixel element, or
dependent upon a highest exceeded threshold value on the respective pixel element and an acquired highest exceeded threshold value on the at least one further pixel element, a coincidence count signal is processed with a mixing factor.

7. The method as claimed in claim 1, wherein at least one of the number of comparators is a switchable comparator, the threshold value of the switchable comparator being settable to different threshold values.

8. A photon-counting X-ray detector, comprising:
at least one converter element connected to a plurality of pixel elements, counters for counting count signals and coincidence counters for counting coincidence count signals,
wherein the at least one converter element is configured to convert incident X-ray radiation into an electrical signal and each pixel element of the plurality of pixel elements is configured to record an electrical signal at a respective position,
wherein each pixel element of the plurality of pixel elements has a number of comparators, each comparator having at least one threshold value,
wherein each comparator of the number of comparators is configured to output an output signal in response to the electrical signal exceeding the threshold value,
wherein the number of comparators includes a first subset of comparators and, in case of at least one subset of the plurality of pixel elements, a second subset of comparators,
wherein each pixel element of the plurality of pixel elements is configured to form at least one count signal based upon the output signal from at least one comparator of the first subset of comparators,
wherein at least the subset of the plurality of pixel elements is configured to form one or more coincidence count signals, each of the one or more coincidence count signals being based upon the output signal generated in a pixel element of the subset of the plurality of pixel elements by a comparator of the number of comparators of the pixel element, and based upon a coinciding output signal generated by a comparator of the number of comparators of at least one further pixel element of the plurality of pixel elements,
wherein the output of each of the comparators of the first subset of comparators is connected directly or indirectly to at least one counter for counting count signals of individual electrical signals, and
wherein the output of each comparator of the second subset of comparators is connected to at least one coincidence counter for counting coincidence signals of coincident electrical signals, and not to a counter for counting count signals of individual electrical signals.

9. An X-ray detector system, configured to carry out the method as claimed in claim 1, the X-ray detector system comprising:
the photon-counting X-ray detector including at least one converter element connected to a plurality of pixel elements,
wherein the at least one converter element is configured to convert incident X-ray radiation into an electrical signal and each pixel element of the plurality of pixel elements is configured to record an electrical signal at a respective position,
wherein each pixel element of the plurality of pixel elements has a number of comparators, each having at least one threshold value,
wherein each comparator of the number of comparators is configured to output an output signal in response to the electrical signal exceeding the threshold value,
wherein the number of comparators includes a first subset of comparators and, in case of at least one subset of the plurality of pixel elements, a second subset of comparators,
wherein each pixel element of the plurality of pixel elements is configured to form at least one count signal based upon the output signal from at least one of the comparators of the first subset,
wherein at least the subset of the plurality of pixel elements is configured to form one or more coincidence count signals, each of which is based upon the output signal generated in a pixel element of the subset of pixel elements by a comparator of the number of comparators of the pixel element, and based upon a coinciding output signal generated by a comparator of the number of comparators of at least one further pixel element of the plurality of pixel elements; and
a generating unit configured to generate an X-ray image dataset based upon a number of count signals counted in each pixel element of the plurality of pixel elements, and based upon a number of coincidence count signals counted in each pixel element of the subset of the plurality of pixel elements.

10. A medical imaging device comprising an X-ray detector system as claimed in claim 9.

11. The method as claimed in claim 3, wherein the threshold value of the at least one comparator of the second subset of comparators is set such that the special information item is better acquired than with threshold values of the first subset of comparators.

12. The method as claimed in claim 4, wherein the threshold value of the at least one comparator of the second subset of comparators is set such that the threshold value of the at least one comparator of the second subset of comparators and a lowest threshold value of the first subset of comparators are on different sides of the fluorescence energy.

13. The photon-counting X-ray detector of claim 8,
wherein the output of each comparator of the first subset of comparators is connected to at least one coincidence counter for counting coincidence signals of coincidental electrical signals.

14. The medical imaging device of claim 10, wherein the medical imaging device is a computed tomography device.

15. The method as claimed in claim 2, wherein
the threshold value of the at least one comparator of the second subset of comparators is set so that a special information item that is relevant for at least one of a scan or examination is acquired in a targeted manner.

16. The method as claimed in claim 15, wherein the threshold value of the at least one comparator of the second subset of comparators is set relative to at least one fluorescence energy of a material of the converter element.

17. The method as claimed in claim 15, wherein for the second counting of the number of coincidence count signals, the threshold value of at least one comparator of a number of comparators of a respective pixel element of the subset of the plurality of pixel elements and the threshold value of at least one comparator of a number of comparators of the at least one further pixel element, upon which the coincidence count signals are based, are set to different threshold values.

18. The method as claimed in claim 2, wherein the threshold value of the at least one comparator of the second subset of comparators is set relative to at least one fluorescence energy of a material of the converter element.

19. The method as claimed in claim 2, wherein for the second counting of the number of coincidence count signals, the threshold value of at least one comparator of a number of comparators of a respective pixel element of the subset of the plurality of pixel elements and the threshold value of at least one comparator of a number of comparators of the at least one further pixel element, upon which the coincidence count signals are based, are set to different threshold values.

20. The method as claimed in claim 2, wherein at least one of the number of comparators is a switchable comparator, the threshold value of the switchable comparator being settable to different threshold values.
